(19) United States
(12) Patent Application Publication
(10) Pub. No.: US 2017/0326176 A1
(43) Pub. Date: Nov. 16, 2017

(54) Epithelial Treatment

(71) Applicant: Anant SHARMA, Bedford (GB)

(72) Inventor: Anant SHARMA, Bedford (GB)

(21) Appl. No.: 15/528,395

(22) PCT Filed: Nov. 24, 2015
(86) PCT No.: PCT/GB2015/000305

§ 371 Date: May 19, 2017

(30) Foreign Application Priority Data
Nov. 21, 2014 (GB) 1420687.4
Nov. 21, 2014 (GB) 1420688.2
Nov. 21, 2014 (GB) 1420689.0

(51) Int. Cl. A61K 35/14; A61K 9/00; A61K 38/01; A61K 8/98; A61K 35/60; A61K 47/38

(52) U.S. Cl. CPC A61K 35/14; A61K 35/60; A61K 9/0048; A61K 38/017; A61K 38/014; A61K 8/983; A61K 9/0014; A61K 47/38

(57) ABSTRACT

Products are described for treating epithelial tissues of the human or animal body including liquid compositions containing whole blood, or parts of blood including blood plasma, blood serum, blood platelets and growth factors derivable from blood. Other compositions disclosed include solid reservoirs of collagen or amniotic membrane into which whole blood or parts of blood have been absorbed. Methods of treatment using these compositions are described, in particular for treatment of ophthalmic disorders such as dry-eye and oral disorders such as dry mouth. A range of devices (1, 11, 31, 41, 51, 61, 71, 81, 91, 101) are described for sampling, mixing, storing and/or applying blood and compositions containing blood and parts of blood.

The present invention relates to a composition for medical treatment of epithelial layers, derived from a patient's own blood. More particularly but not exclusively, it relates to such a composition for treatment of ophthalmic conditions. It may further relate to methods of treatment using such compositions and to apparatus usable in the preparation and administration of such compositions.

A surface layer of the human and animal body is made up of specialised cells, known as epithelial cells, forming the epithelial layer or epithelium. There are numerous conditions which are common to most epithelial cells, and which are often treatable using similar approaches. Some epithelial cells are even more specialised, however, a particular example being those forming an outer layer of the eye, which are classed as non-keratinising epithelial cells. Thus, ophthalmic disorders and surface damage may in some cases be treatable by techniques and substances that are also usable on the epithelium in general (e.g. the skin, mouth), but in other cases may require specialist treatments.

It is known to treat a range of disorders of the human or animal eye using wetting and lubricating solutions, known as “artificial tears”. Such artificial tears contain a range of water-soluble viscosity modifiers and wetting agents, to ensure that they cover the ocular surface and remain for prolonged periods, in order to make up for deficiencies in the quantity or quality of “natural” tears, for example. A condition known variously as dry eye syndrome, keratoconjunctivitis sicca, xerophthalmia or simply “dry eye” leaves the eye deficient in lubrication and leads to redness, itching, and pain (and possibly damage to the eye if adventitious pollutants and particulates were to contact the eye without promptly being diluted and washed away by tears). Artificial tears are also used to alleviate the problems resulting from corneal ulcers or other conditions involving damage to the epithelial layer of cells of the eye. In each case, however, such artificial tears are a stand-in for “natural” tears and generally do not contribute directly to active healing of the underlying condition.

Where healing does not occur as a result of the body's own resources, merely alleviating symptoms with artificial tears is insufficient. One approach that has been used with some success is to boost healing locally by the administration of natural healing factors. To avoid immunological compatibility issues, these may be derived from a patient's own blood. “Autologous serum eyedrops” are produced by processing whole blood, taken from the patient by the same procedures as are used for blood donation. The whole blood is treated to separate red blood cells from the blood serum, for example by coagulation and/or centrifugation, and the serum is then diluted with saline solution and dispensed into multiple storage containers. The patient, or a carer, administers the autologous serum eyedrops to the eye, where the various proteins, enzymes, healing factors and growth factors naturally present in the serum assist healing of the underlying condition, supplementing the same compounds being delivered to the eye through the patient's bloodstream and natural tears.

There are practical problems, however. Processing the whole blood to produce serum free from blood cells is a lengthy process. It is hence only worthwhile when carried out on a significant quantity of blood at once, producing sufficient eyedrops for several month's use. Precautions must also be taken to ensure that the correct eyedrops are supplied to each patient. To ensure the eyedrops remain fresh and sterile, each month's supply is delivered to the patient in a plurality of small bottles. The eyedrops are delivered frozen and must be kept frozen, to ensure sterility and to avoid decomposition of less stable components. One bottle at a time must be allowed to thaw to provide the next day's course of eyedrops. This involves significant organisation and planning, and is far from convenient for the patient.

Additionally, there are suggestions that the high speed centrifugation employed to separate red blood cells may also reduce the content of high molecular weight growth factors and other proteins in the supernatant serum.

It is hence an object of the present invention to provide a composition, administrable to the surface of the eye and possibly to other epithelial cells, that provides the above benefits while obviating at least some of the above problems. It is also an object of the invention to provide methods of production and administration of such compositions. Another object of the invention is to provide equipment to aid the production, storage and/or administration of such compositions.

According to a first aspect of the present invention, there is provided a composition for the treatment of epithelial disorders for a particular human or non-human animal patient, comprising autologous whole blood extracted from said patient, mixed with an aqueous carrier medium.

Preferably, the composition is adapted for the treatment of ophthalmic disorders.

Advantageously, the composition is adapted for the treatment of dry eye syndrome, keratoconjunctivitis sicca, xerophthalmia, corneal ulcers and/or physical damage to the surface of the eye.

The aqueous carrier medium may comprise an eyedrops formulation.

The aqueous carrier medium may comprise an artificial tears composition.

The aqueous carrier medium may comprise one of more of hypromellose, carbomer gel, carmellose and sodium hyaluronate solutions.

The aqueous carrier medium may comprise a preservative reagent, optionally a bacteriostat.

The aqueous carrier medium may comprise cell nutrient reagents adapted to maintain the viability of red and white blood cells and platelets therein.

The aqueous carrier medium may be isotonic with blood.

The aqueous carrier medium may have a pH between pH6 and pH8.

The aqueous carrier medium may comprise an anticoagulant reagent, such as heparin, adapted to retard or obviate coagulation of red and white blood cells and blood platelets suspended in the carrier medium.

The aqueous carrier medium may comprise a stabilising reagent adapted to retard denaturing and coagulation of proteins in the blood.

Alternatively, the aqueous carrier medium may comprise a coagulation agent adapted to coagulate red and white blood cells and/or blood platelets in order to encourage their separation from the composition before use.

The aqueous carrier medium may comprise a gel medium or a medium adapted to gel on application to the epithelial layer.

The composition may comprise an ointment adapted for direct application to a surface of the epithelial layer.

The aqueous carrier medium may comprise a medium adapted for injection into a patient's body.

According to a second aspect of the present invention, there is provided a composition for the treatment of epithelial disorders for a human or non-human animal patient, comprising growth factors or proteins, mixed with an aqueous carrier medium.

Said composition may comprise features corresponding to any or all of the features of the first aspect above.

According to a third aspect of the present invention, there is provided a method for the treatment of epithelial disorders of humans or non-human animals, comprising the steps of extracting a quantity of whole blood from a patient requiring treatment for said disorder, mixing said whole blood with an aqueous carrier solution to form a treatment composition and administering said treatment composition to or adjacent disordered epithelial tissues of the patient.

Preferably, the method is a method for treatment of disorders of the eye.

Said disorders of the eye may be selected from a list comprising dry eye syndrome, keratoconjunctivitis sicca, xerophthalmia, corneal ulcers and physical damage to the surface of the eye.

Preferably, said administration step is carried out promptly after said mixing step.

Advantageously, at least one further administration step is carried out with said treatment composition, the or each further administration step following a respective storage step, optionally under refrigeration.

Optionally, said blood extraction step, said mixing step, said administration steps and the or each storage step may be carried out within a day.

Alternatively, an initial storage step may take place between the mixing step and a first administration step.

The method may comprise at least one preservation step.

Said preservation step may comprise incorporating a preservative agent in the aqueous carrier solution, or its separate admixture.

Said preservation step may comprise incorporating one or more cell nutrient reagents in the aqueous carrier solution, or their separate admixture.

Said preservation step may comprise incorporating an anticoagulant reagent in the aqueous carrier solution, or its separate admixture.

Said preservation step may additionally or alternatively comprise a filtration step to remove cellular material, such as bacterial cells.

Said filtration step may precede or follow the mixing step.

Said filtration step may be performed with filter means having a mean effective pore diameter of less than 1 μm, optionally of less than 0.3 μm.

Said preservation step may additionally or alternatively comprise carrying out said initial storage step under refrigeration.

The blood extraction step may comprise pricking a part of the patient's body having a plentiful blood supply adjacent a surface of the body, such as a fingertip or an earlobe, and collecting a drop of whole blood produced therefrom.

Alternatively, the blood extraction step may comprise taking a sample of venous blood from the patient.

In a first embodiment, said treatment composition comprises an artificial tears or eyedrops composition, and the application step comprises instilling the composition on to a surface of the eye.

The aqueous carrier solution may then comprise one or more of hypromellose, carbomer gel, carmellose and sodium hyaluronate solutions.

In a second embodiment, said treatment composition comprises an ointment or gel adapted for topical application to an epithelial surface, and the application step may then comprise application of the composition to or adjacent damaged or disordered epithelium.

In a third embodiment, said treatment composition comprises an injectable composition.

The administration step may then comprise injection of the treatment composition into body tissues adjacent damaged or disordered epithelium.

According to a fourth aspect of the present invention, there is provided a method for the treatment of epithelial disorders of humans or non-human animals, comprising the steps of providing one or more growth factors or proteins, mixing said growth factors or proteins with an aqueous carrier solution to form a treatment composition and administering said treatment composition to or adjacent disordered epithelial tissues of the patient.

Preferably, said method comprises steps corresponding to any or all of the steps of the method of the third aspect above.

According to a fifth aspect of the present invention, there is provided a storage and mixing vessel adapted to be used in a treatment method as described in the third aspect, above, comprising first chamber means containing an aqueous carrier solution miscible with whole blood to form a treatment composition, and means to introduce said whole blood for mixing.

In a first embodiment, said vessel comprises second chamber means separated from the first by frangible divider means, the blood being introducible into the second chamber means such that subsequent breach of the divider means permits mixing of the blood and the aqueous carrier solution.

In a second embodiment, said vessel comprises filter means or semipermeable membrane means.

Said filter means may have an effective mean pore diameter of 1 μm or below, optionally of 0.3 μm or below.

Said filter means may be so disposed as to filter whole blood introduced into the vessel.

Said filter means may be so disposed as to filter the treatment composition as it is dispensed from the vessel.

In a third embodiment, the vessel is provided with sealing cap means comprising septum means penetrable by hypodermic syringe means to allow extraction of the treatment composition from the vessel.

Preferably, the septum means is also penetrable by hypodermic syringe means to deliver whole blood into the vessel for mixing.

The vessel has a known volume and several bottles can be prepared.

According to a sixth aspect of the present invention, there is provided a treatment element for treating disorders of epithelial tissues of the human or non-human animal body, comprising solid reservoir means of collagen or amniotic membrane containing a dose of blood or a blood product.

Preferably, the treatment element is for treatment of a particular human or non-human animal patient, and said blood comprises autologous whole blood from said patient.

Alternatively, the treatment element is for treatment of a non-specific patient and said blood product comprises blood serum.

Alternatively, the blood product may comprise one or more growth factors isolable from blood.

In a first embodiment, the treatment element is for treatment of tissues of the eye, and the reservoir means is adapted to be located in the lower fornix of the eye of the patient.

The reservoir means may then comprise generally cylindrical rod means, less than 15 millimetres in length and less than 4 millimetres in diameter.

The generally cylindrical rod means may be no more than 5 millimetres in length and no more than 2 millimetres in diameter.

In a second embodiment, the treatment element is for treatment of tissues of the eye, and the reservoir means comprises a punctal plug, adapted for insertion into a tear duct.

The reservoir means may then comprise generally cylindrical rod means, less than 3 millimetres in length and less than 1 millimetre in diameter.

In a third embodiment, the treatment element is for treatment of tissues of the mouth, and the reservoir of collagen or amniotic membrane has the form of a generally spheroidal or ellipsoidal lozenge or pastille.

The lozenge may then be between 5 and 15 millimetres in average diameter.

The treatment element may advantageously be for treatment of dry mouth.

In a fourth embodiment, the treatment element is for treatment of tissues of the mouth, and the reservoir of collagen or amniotic membrane has the form of a pad or patch contactable with said tissues.

The reservoir of collagen or amniotic membrane may then be generally laminar with a maximum thickness of less than 5 millimetres, optionally less than 3 millimetres.

The treatment element may advantageously be for treatment of mouth ulcers, the reservoir of collagen or amniotic membrane being configured to cover an entire area of ulceration.

Alternatively, the reservoir of collagen or amniotic membrane may be configured to cover a single ulcerated lesion.

In a fifth embodiment, the treatment element is for treatment of skin tissues, and the reservoir of collagen or amniotic membrane has the form of a dressing or bandage.

The reservoir of collagen or amniotic membrane may then be generally laminar with a maximum thickness of less than 3 millimetres, optionally less than 2 millimetres.

Preferably, said collagen comprises collagen of non-human origin.

Advantageously, said collagen comprises collagen from an aquatic organism, optionally derived from jellyfish or other cnidarians.

Said collagen may comprise fragmented collagen or hydrolysed collagen.

Said collagen may comprise collagen sponge or collagen gel.

Said amniotic membrane may comprise amniotic membrane of non-human origin.

According to a seventh aspect of the present invention, there is provided a method of treatment for disorders of epithelial tissues of the human or non-human animal body, comprising the steps of providing a solid reservoir of collagen or amniotic membrane; providing a dose of blood or blood product; absorbing said dose into the reservoir of collagen or amniotic membrane; positioning the reservoir of collagen or amniotic membrane adjacent the tissues to be treated; and allowing the blood or blood product to diffuse or disperse out of the reservoir of collagen or amniotic membrane to contact said tissues.

The method of treatment may comprise treatment of a particular human or non-human animal patient, said dose of blood comprising autologous whole blood.

Said autologous whole blood may then be provided by extemporaneous collection, optionally by pinprick or finger-prick collection for example with lancet means.

The method of treatment may be for treatment of a non-specific patient, said dose of blood product comprising blood plasma or serum, preferably serum.

Said blood serum may then have previously been produced by treatment of heterologous whole blood, optionally followed by a storage step, for example a frozen storage step.

Said blood serum may alternatively be produced by treatment of autologous blood.

According to an eighth aspect of the present invention, there is provided a composition for the treatment of disorders of epithelial tissues of the mouth for a human or non-human animal patient, comprising blood or parts of the blood or blood products incorporated into a preparation adapted for application to the epithelial tissues of the mouth.

Preferably, said blood comprises whole blood.

Said parts of the blood may comprise blood plasma, blood serum and/or blood platelets.

Said blood products may comprise growth factors isolable from blood.

Said blood or parts of the blood or blood products may be of human origin.

Alternatively, said blood or parts of the blood or blood products may be of non-human animal origin.

Said preparation may comprise a liquid.

Said preparation may comprise mouthwash means.

Said mouthwash means may comprise collagen.

Said collagen may comprise collagen dispersed or dissolved in water.

Said collagen may comprise collagen gel.

The mouthwash means may comprise a viscosity modifier and/or gelling agent.

The mouthwash means may then be adapted to be retained on a epithelial tissue surface.

Said disorders may comprise dry mouth syndrome.

Said disorders may comprise ulcers, including iatrogenic ulcers, for example resulting from cancer treatments.

According to a ninth aspect of the present application, there is provided a method of treatment for disorders of the epithelial tissues of the mouth for a human or non-human animal patient, comprising the steps of providing a composition as described in the eighth aspect above and applying it to an interior of the mouth so as to contact the tissues to be treated.

Preferably, the method comprises the step of applying a composition of sufficient viscosity or gel strength as to coat and be retained on the interior of the mouth after application.

According to a tenth aspect of the present invention, there is provided a composition for the treatment of disorders of epithelial tissues for a human or non-human animal patient, comprising dehydrated blood or parts of the blood from which water has been partially or totally removed.

Said removal of water may be by spray drying.

Said removal of water may be by freeze-drying or lyophilisation.

Preferably, said dehydrated blood or parts of blood are adapted to be rehydrated for use in the treatment.

Said blood or parts of the blood may comprise any or all of whole blood, blood plasma, blood serum and/or blood platelets.

According to a eleventh aspect of the present invention, there is provided a blood collection and application device, the device comprising flexible wall means defining internally chamber means containing an aqueous carrier solution miscible with blood or a blood product to form a treatment composition, and means to introduce said bodily fluid into said chamber means for mixing.

According to a twelfth aspect of the present invention, there is provided a blood collection and application device, the device comprising elongate outer casing means including a resiliently compressible portion, the casing means defining internally a cavity; and flexible chamber means located within said cavity and comprising tube means extending from said chamber means outwardly through said outer casing means, said tube means communicating said chamber means with a surrounding environment; in which said flexible chamber means is movable along said cavity between a first position and a second position, wherein in said second position a majority of the tube means is disposed within the outer casing means and wherein in said first position, said flexible chamber is positioned proximal said resiliently compressible portion of said outer casing.

According to a thirteenth aspect of the present invention, there is provided a blood collection and application device, the device comprising hollow elongate rigid tube means having a first open end and a second end, and having pellet means of collagen or amniotic membrane located within said tube means adjacent said first open end; and plunger means located within said tube means comprising a head portion and a shaft portion, said head portion being contactable with said pellet means and said shaft portion extending outwardly from said second end of said tube; in which said plunger means is slidable within said tube means, from a first position towards a second position, and wherein in said second position, said head portion causes said pellet means to be displaced from within said tube.

According to a fourteenth aspect of the present invention there is provided a blood collection and application device, the device comprising hollow elongate compressible tube means having a first open end and a second closed end, and having pellet means of collagen or amniotic membrane located within said tube means adjacent said first open end; in which compression of said compressible tube means urges said pellet to eject from within said tube means.

According to a fifteenth aspect of the present invention, there is provided a blood collection and application device, the device comprising elongate rigid rod means having a first end and a second end, the rod means having pellet means of collagen or amniotic membrane releasably attached proximal said first end; in which said pellet means is disengageable from said rod by an external force incident said rod means or said pellet means.

According to a sixteenth aspect of the present invention, there is provided a blood collection and application device, the device comprising an elongate main body defining an adsorption surface, in which said adsorption surface is so treated or configured as to attract blood to flow on to said adsorption surface.

Preferably, said adsorption surface is coated with one or more reagents such as a lubricant, anti-bacterials, anti-coagulants, coagulants, stabilising agents, and/or collagen to mix with the blood

Embodiments of the present invention will now be described by way of example and with reference to the accompanying drawings, in which:

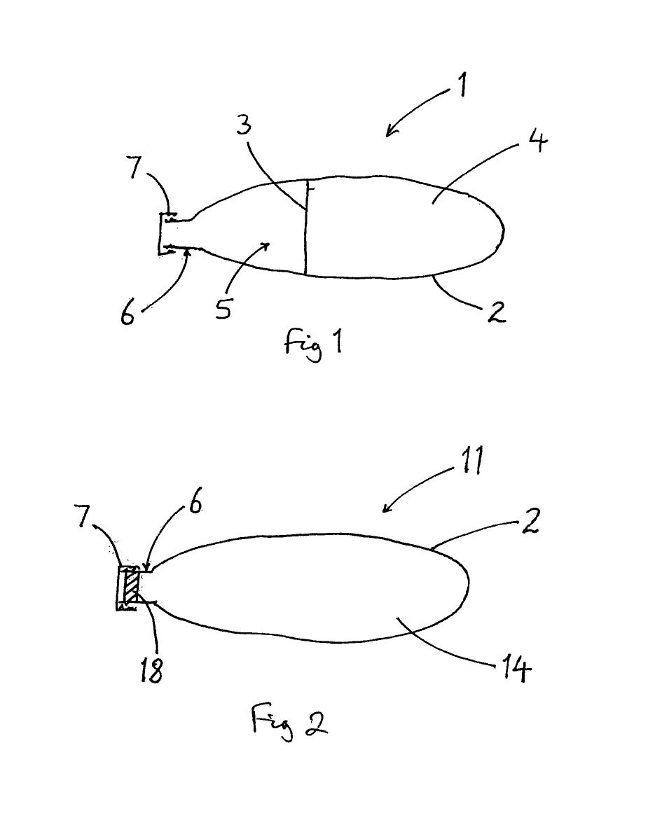

FIG. 1 is a schematic longitudinal cross-section of a first sampling and storage container embodying the present invention;

FIG. 2 is a schematic longitudinal cross-section of a second sampling and storage container embodying the present invention;

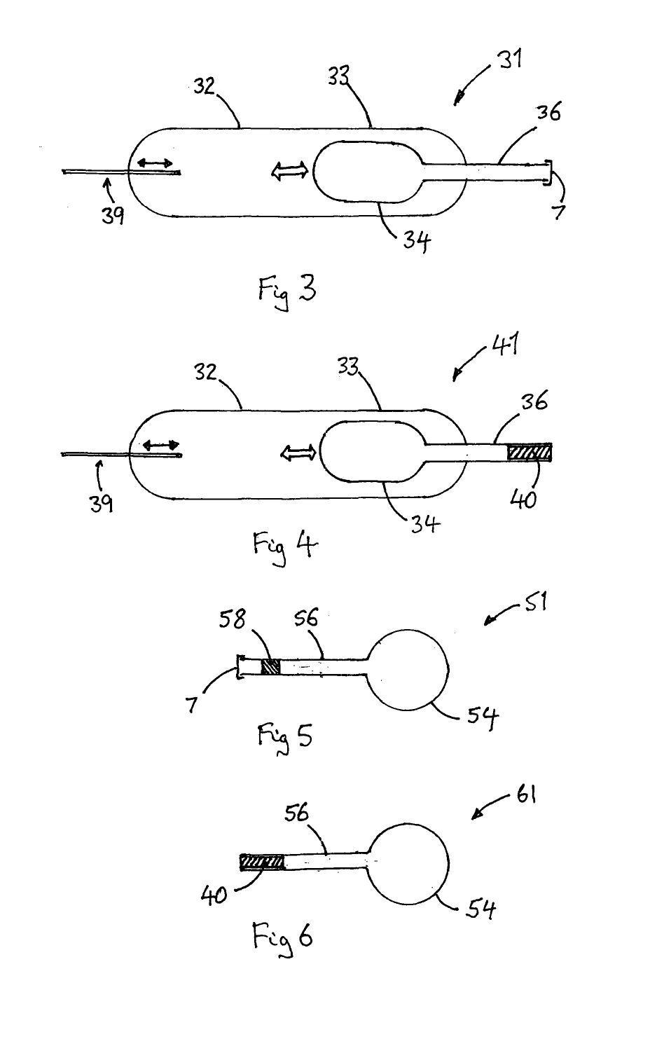

FIG. 3 is a schematic longitudinal cross-section of a first collection and application device embodying the present invention;

FIG. 4 is a schematic longitudinal cross-section of a second collection and application device embodying the present invention;

FIG. 5 is a schematic longitudinal cross-section of a third collection and application device embodying the present invention;

FIG. 6 is a schematic longitudinal cross-section of a fourth collection and application device embodying the present invention;

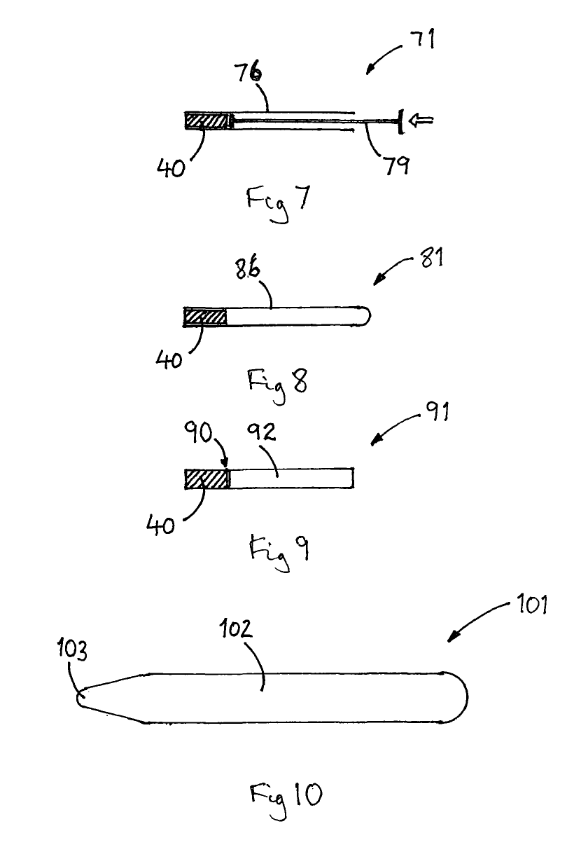

FIG. 7 is a schematic longitudinal cross-section of a fifth collection and application device embodying the present invention;

FIG. 8 is a schematic longitudinal cross-section of a sixth collection and application device embodying the present invention;

FIG. 9 is a schematic longitudinal cross-section of a seventh collection and application device embodying the present invention; and

FIG. 10 is a schematic longitudinal cross-section of a eighth collection and application device embodying the present invention.

In a first example, whole blood was taken from a patient suffering from dry eye syndrome, by a conventional finger prick technique (blood from any source may be employed, including venous blood taken by phlebotomy, or any internal or external wound or sampling technique, as long as contamination by non-blood material may be avoided).

The drop of blood produced was sucked up into an open 1 ml plastic sample vial of conventional form, containing 0.5 ml of 0.1% w/v, aqueous sodium hyaluronate. The vial was sealed and the contents were mixed by gentle shaking. The eyedrop solution produced was administered to an eye of the patient, alleviating the symptoms of the dry eye syndrome for in excess of one hour.

The eyedrop solution described was sufficient for four applications to the eye over the course of a day, following which the vial was discarded. The preparation and administration of the whole blood eyedrop solution was repeated daily, as described above, reducing discomfort and producing a qualitative reduction in the severity of the dry eye syndrome. Osmolarity readings also fell, which is associated with alleviation or healing of the dry eye syndrome.

In a second example, whole blood was again taken from a patient suffering from mild dry eye syndrome by the finger prick technique (for convenience). The blood drop was sucked up into an open plastic bottle containing 0.5 ml aqueous chloramphenicol (chloramphenicol is a bacteriostatic antimicrobial, used inter alia to halt the progress of eye infections and as a preservative medium). The bottle was sealed and its contents were mixed by gentle shaking. The eyedrop solution produced was administered to the eye of the patient as a small drop. The bottle was stored in a refrigerator at around 4° C. (conventional domestic refrigeration suffices) and was removed for periodic further administration of the eyedrops during the day of their production. Again, the symptoms of dry-eye syndrome were alleviated, to a degree significantly greater than would result from administration of a course of 0.5% chloramphenicol solution alone.

In a third example, whole blood taken as a finger prick from a patient with dry eye syndrome was sucked up into a 1 ml aliquot of saline solution in a vial, and mixed by gentle shaking. The eyedrops were administered several times during the day of production. In this example, it proved necessary to shake the sealed vial before each administration to reverse any settlement or other forms of separation. Surprisingly the blood did not coagulate.

Plain saline solution is not usually considered an optimum material for optical lubrication/“artificial tears”. The full range of lubricants/viscosity modifiers used in conventional artificial tears may be used in the present invention, such as hypromellose (hydroxypropyl methylcellulose), carbomer gel (high molecular weight polyacrylates, usually cross linked with a polyol), carmellose (carboxymethyl cellulose) and sodium hyaluronate (a natural or synthetic high molecular weight glycosaminoglycan, which is naturally present in epithelial cells, and so is particularly suitable for use in epithelial treatments). These “artificial tears” of conventional form are particularly suitable for mixing with autologous whole blood to form the eyedrops of the present invention, since they lubricate the eye and have similar rheology to natural tears, ensuring that they remain spread across the surface of the eye for long enough for the healing components of the blood to act on the epithelial cells. Indeed, it may be beneficial to arrange for the artificial tears to have a higher viscosity than natural tears, to maximise residence time. It is known to formulate artificial tears with high levels of sodium hyaluronate such that they gel slightly on administration to the eye, further increasing the residence time of the active components of the eyedrops (i.e. in the present case, the active components of the blood).

In many embodiments of the present invention, a specialised first vial 1 may be used, as shown in FIG. 1. The first vial has a flexible outer wall 2, made from a plastics material, and is divided by a frangible seal 3 into a distal compartment 4 containing a desired quantity of a sterile eyedrop composition and a proximal compartment 5, into which a blood drop (or blood from a larger sample) may be drawn through a narrow neck 6. The neck 6 may be sealed with a cap 7 of conventional form. Once sealed, the first vial 1 is squeezed to break the seal 3 and allow mixing of the blood with whatever composition is held within the distal compartment 4. The blood-containing eyedrops produced are simple to administer by removing the cap 7 and dispensing one or more drops through the neck 6 by squeezing the outer wall 2.

In a further variant of the production method, a microfilter is used to separate dissolved proteins, growth factors and the like in the blood serum from the red and white blood cells. It is not yet known whether red or white blood cells and platelets in particular have a beneficial effect in the present invention. Production of serum by conventional coagulation and settlement/centrifugation techniques is regarded as too time consuming and uneconomic for daily use on the sub-millilitre scale. However, at this “blood drop” scale, passing the whole blood through a filter having a pore size of around 0.2 μm appears to be a straightforward approach to separating the cells from the serum. The whole blood may be filtered in this way as it is taken up into a vial or other mixing/storage vessel, or the eyedrops, etc, containing whole blood may be dispensed from the vessel through such a filter.

Using the filter as the blood is taken up into the vial, etc, maybe beneficial as capillary effects may be used to control the rate or volume of blood taken up.

An example of such a second vial 11 is shown in FIG. 2. As for the first vial 1, the second vial 11 has a flexible outer wall 2, in this case defining an internal chamber 14 holding a desired quantity of an eyedrop/artificial tears composition. The second vial 11 is also provided with a narrow neck 6 sealable with a cap 7, but in this case the neck 6 contains a micro filter 18. Thus, blood taken up into the chamber 14 through the neck 6 must pass through the filter 18, with the result that only blood serum enters to mix with the eyedrops/artificial tears composition within the chamber 14. Squeezing the wall 2 of the second vial 11 delivers the blood serum/eyedrops/artificial tears composition out through the filter 18 in the neck 6.

Alternatively, the filter 18 could be mounted to the neck 6 only once the whole blood had been mixed into the contents of the internal chamber 14. Thus, red and white blood cells and platelets would be retained within the chamber 14 by the filter 18, and the eyedrops/artificial tears dispensed would in effect contain only the blood serum.

Such sub-micron filters also have the benefit of filtering out most or all bacteria. Thus, compositions prepared and stored in such vials should remain usable for longer, even if preservatives and/or refrigeration are not used. Semi-permeable membranes of other types having similar properties may also be used.

A range of other additives may be incorporated into the solutions held within the various mixing bottles and vials. For example, it may be desirable to maintain red and white blood cells and blood platelets in suspension in the whole blood eyedrops, particularly if for any reason the eyedrops are to be stored for more than a few hours. In such cases, the solution to be mixed with the autologous whole blood would preferably contain heparin or other anticoagulant, to retard coagulation and irreversible settlement/separation of the cells and platelets. Heparin, etc, should also retard denaturing and coagulation of high molecular weight proteins, enzymes and growth/healing factors.

Additionally or alternatively, in order to ensure long-term viability of blood cells, enzyme and so forth in the eyedrops, the solutions to be mixed with the whole blood should be isotonic with the blood serum, and should have a compatible pH, ideally in the range of pH6-8. Cell nutrients may also be included for prolonged storage (while serum components should survive cold storage, cells would usually die in a few days). In such systems, care should be taken to avoid microbiological contamination, so that bacterial proliferation is avoided. Chloramphenicol may be useful in such systems, as it acts as a bacteriostat but appears not to harm the blood cells.

If it is desired to remove blood cells and platelets from a composition, in order to produce a composition approximating to autologous serum eyedrops, then known coagulants may be incorporated in the solution to be mixed with the whole blood.

As well as eyedrops and artificial tears, similar approaches can be used to produce ointments and injectable compositions. Injectable compositions may be required when epithelial damage goes deep, for example. A conventional injectable carrier solution, such as sterile saline solution, may be mixed with whole blood in a vial provided with a septum cap, such that the mixed composition may be withdrawn into the syringe by passing a hypodermic syringe needle through the septum, while maintaining the sterility of the composition left in the vial.

Such a septum cap may also permit whole blood taken up into a syringe to be delivered into the vial without opening the cap and risking contamination.

Although injectable compositions should generally be administered by a medical professional, it would still be beneficial if the compositions could be made up on an ad hoc basis, or if the patient or a carer could produce a stock of autologous blood composition ready for professional administration.

A major benefit of the present invention is clearly the ability of a patient or carer to make up his or her own eyedrops, etc, as required. However, it is also envisaged that the same method and compositions could be used on a larger scale, particularly using a venous blood sample, in order to produce a batch of autologous whole blood eyedrops to last for several days or even weeks and months. It is simply necessary to employ the various chemical and physical treatments discussed above in order to ensure that the eyedrops have a constant composition and acceptable sterility throughout the batch.

Eyedrops, etc, may also be stored in sealed sterile containers and withdrawn using a conventional syringe and hypodermic needle passed through the septum, before being carefully dripped on to the eye (typically after removal of the needle from the syringe). the ‘syringe is the bottle’ or becomes part of the bottle.

Where a syringe or dropper is used to deliver the eyedrops, artificial tears, etc, to the eye, a microfilter or other semipermeable membrane may be mounted to the syringe, rather than being associated with the vial or other storage/mixing vessel.

The present invention is believed to comprise a significant improvement on existing approaches using autologous serum eyedrops manufactured from a patient's whole blood by a range of complex separation methods. These are summarised in the UK National Health Service patient information document “Autologous Serum Eyedrops” (reference INF/PTI/PR/009/01;08/04), and articles in their use include “Autologous serum eye drops for ocular surface disorders”, G Geerling, S MacLennan & D Hartwig, Br. J Ophthalmol, 2004; 88; 1467-1474; “Comparison of autologous serum eye drops with conventional therapy in a randomised controlled crossover trial for ocular surface disease”, BA Noble, RSK Loh, S MacLennan, K Perudovs, A Reynolds, L R Bridges, J Burr, 0 Stewart & S Quereshi; Br. J Ophthalmol, 2004; 88; 647-652; and “Autologous serum eyedrops for dry eyes and epithelial defects: clinical and in vitro toxicity studies”; A C Poon, G Geerling, J K G Dart, G E Fraenkel & J T Daniels, Br. J. Ophthalmol; 2001; 85; 1188-1197. An initial contribution towards addressing issues with such serum eyedrops was reported in “Simple finger prick fresh blood technique for use on the ocular surface”; A Sharma, BAR Sharma, J Moore & M Nolan; Contact Lens & Anterior Eye; 34 (2011); 49.

While the present invention is described above in terms of its use on human patients, non-human animals and birds may be affected by similar conditions. Animal epithelial and ophthalmic structure is very similar to human, at the level addressed by the present invention. Thus, autologous veterinary eyedrops, injectable compositions and the like may be made up using blood taken from an affected animal and administered to that animal, in substantially the same manner.

Further devices for use in the present invention will now be described.

FIG. 3 shows a first blood collection and application device 31. This has an elongate outer casing 32 enclosing a flexible/resilient chamber or bulb 34. An elongate neck or tube 36 extends from the chamber 34 outwardly through a first end of the outer casing 32, and is sealed with a removable cap 7 of conventional form. The tube 36 can be extended or retracted by pulling or pushing it longitudinally; the chamber 34 moves accordingly along the outer casing 32, as shown by the double arrow. A retractable lancet 39 extends, in use, from a second end of the outer casing 32 remote from the first. Either the outer casing 32 as a whole comprises a resiliently deformable material, or at least a compressible portion 33 of the outer casing 32 adjacent the chamber 34 (when the tube 36 is extended) is resiliently deformable. The compressible portion 33 may comprise a resilient material and/or may have a resilient construction, such as resiliently mounted hinged panels. In each case, the compressible portion 33 may be squeezed or crushed manually so as to compress or release pressure on the chamber 34 as desired. Alternatively, the portion 33 of the casing 32 may comprise removable or retractable panels to give direct access to the chamber 34 for squeezing.

The first device 31 is used as follows. The lancet 39 is extended and is used to prick the user's finger or thumb to produce a drop of fresh blood. (Other parts of the body might be used, but the tips of the fingers and thumbs very readily produce a drop of blood sufficient for testing or other purposes, without excessive subsequent bleeding; this technique of producing a sample of fresh blood is widely known as the finger-prick or finger-stick method as a result). The cap 7 is removed, and the drop of blood is sucked up into the tube 36. It may be taken up into the tube 36 by capillary action if the tube 36 is narrow enough, but more reliably, the chamber 34 may first be squeezed through the squeezable portion 33 of the outer casing 32, producing suction up the tube 36 when the chamber 34 is released, which sucks up the blood.

Depending on the particular method in which the first device 31 is to be used, it may simply retain the blood, which is then expelled when required, for example into the user's own eye to treat conditions such as dry eye or ulceration. Alternatively, the chamber 34 may already contain solid or liquid materials and reagents to mix with the blood. This may range from simple sterile saline solution for dilution to produce eye-drops to more complex “artificial tears” formulations of conventional form, based on hypromellose, carbomer gel, carmellose and/or sodium hyaluronate solutions. Anti-bacterial agents may be present if the blood is not to be used immediately, as may an anticoagulant such as heparin (to retard coagulation of red and white blood cells and blood platelets) and/or a stabilising agent to retard denaturing and coagulation of blood proteins, and/or even a coagulation agent to coagulate red and white blood cells and platelets, thereby potentially rupturing them to release growth factors into the surrounding serum. Dissolved or dispersed collagen may be present to raise viscosities of the mixtures.

Whichever of these materials is held in the chamber 34, it can be stored safely for significant periods, as long as the cap 7 remains hermetically sealed.

In all cases, the blood and reagents stored in the chamber 34 can be mixed by kneading the chamber 34. The mixture can then be delivered to the tissues to be treated by compressing the chamber 34 sufficiently sharply to expel the mixture, out through the tube 36.

The device 31 and those described below are expected to be disposable, single-use equipment, for the sake of hygiene.

In FIG. 4, a second blood collection and application device 41 is shown, which is very similar to the first device 31 of FIG. 3. There is an outer casing 32 with a compressible portion 33, holding a flexible/resilient chamber or bulb 34. An elongate tube 36 extends from the chamber 34 and out through a first end of the casing 32, and can be slid in and out of the casing 32, thus also sliding the chamber 34 within the casing 32 as shown. A retractable lancet 39 is extendable from a second end of the casing 32 remote from the first.

The difference between the first 31 and second 41 devices is the presence of a cylindrical pellet or plug 40 of collagen, amniotic membrane or the like, held snugly within an end of the tube 36 external to the casing 32.

A fresh blood drop is produced with the lancet 39, as for the first device 31. This blood will soak up directly into the pellet 40 as soon as the end of the tube 36 is brought into contact with the blood. The blood-soaked pellet 40 may be expelled pneumatically by pressure on the chamber 34 via the compressible portion 33 of the casing 32. Gentle pressure may express the pellet 40 slowly and gently enough for direct delivery to the site of treatment—e.g. deposition in the lower fornix of the eyelid as a slow-release reservoir. Alternatively, the pellet 40 may be blown out into another container, from which it is collected for application to the tissues, e.g. by conventional methods for inserting a punctual plug into a tear duct, again to act as a slow-release reservoir.

If it is desired that the fresh blood should be diluted or mixed with reagents before it is absorbed into the collagen, etc of the plug/pellet 40, the relevant materials may be held within the chamber 34, as described for the first device 31 above. The plug/pellet 40 would then not be inserted into the end of the tube 36 until after the blood had been taken up into the chamber and mixed. The mixed/treated blood would then be displaced back down the tube 36 until it reached and soaked the collagen, etc, of the plug/pellet 40.

To load larger bodies of collagen, etc, with blood or treated/mixed blood, such as those for use on mouth ulcers or leg ulcers, the blood can be taken up and mixed (as necessary) in the first device 31, then squeezed out on to the body of collagen, sufficient blood being taken up into the chamber to fully load the body with the blood, or treated/mixed blood.

FIG. 5 shows a third blood collection and application device 51, similar to, but simpler than the first device 31. This device 51 comprises a resilient bulb 54, from which extends an elongate tube 56, sealed with a cap 7 when not in use. This particular example of the third device 51 is provided with a filter 58 within the tube 56 (NB such filters could also be applied to the first device 31 if desired). In one variant, the filter 58 acts as a simple relatively coarse filter to exclude macroscopic pollutants from the remainder of the tube 56 and the bulb 54. Alternatively, a microporous filter 58 can be used to exclude particles in the micron range and below, such as bacteria, or even to separate red and white blood cells, and even platelets, from the blood serum, if the treatment method in use so requires.

In use without the filter 58, the third device 51 must be used in conjunction with a conventional blood lancet to form the droplet of fresh blood. Apart from that (and the absence of an outer casing 32/33 so that the bulb 54 may be compressed directly), the third device 51 may be used like the first 31; blood may be taken up, mixed or treated with materials held in the bulb 54 as they were in the chamber 34, and delivered as desired.

FIG. 6 shows a fourth blood collection and application device 61, related to the second device 41 as the third device 51 is related to the first device 31. As with the second device 41, a cylindrical pellet or plug 40 of collagen, amniotic membrane or the like is held in the end of the elongate tube 56.

Apart from needing a droplet of fresh blood to be formed with a conventional blood lancet, and apart from the absence of the casing 32/33, allowing direct manipulation of the bulb 54, the fourth device 61 is used in exactly the same manner as the second device 41.

FIG. 7 shows a fifth blood collection and application device 71, specifically intended for soaking a pellet or plug 40 of collagen with fresh blood.

The fifth device 71 comprises a hollow elongate rigid tube 76, with a cylindrical plug or pellet 50 of collagen, amniotic membrane or the like held within a first end of the tube 76. A plunger 79 extends longitudinally within the tube 76, contacting an inner end face of the plug 40. The plunger 79 extends outwardly from a second end of the tube 76 remote from the first.

In use, a drop of fresh blood is produced by a conventional finger-prick using a conventional blood lancet. The first end of the tube 76 and the enclosed plug/pellet 40 are brought into, contact with the drop of blood, which soaks into the collagen, etc of the plug/pellet 40.

Pressure on the plunger 79 then pushes the plug/pellet 40 out of the tube 76. It may be possible with sufficiently careful manipulation of the plunger 79 to express the plug/pellet 40 directly to the location to be treated, e.g. the lower fornix of the eyelid to act as a slow release reservoir, releasing blood into the eye. Alternatively, the pellet 40 may simply be pushed out of the tube 76 into another container, from which it is picked up and delivered to a desired point of application using existing tools and methods.

An even simpler sixth blood collection and application device 81 is shown in FIG. 8. This is also for use in soaking a pellet or plug 40 of collagen with fresh blood. The sixth device 81 comprises an elongate, hollow, resilient tube 86, with a cylindrical plug or pellet 40 of collagen, amniotic membrane or the like held within an open first end of the tube 86. A second end of the tube 86 remote from the first is closed.

In use a conventional finger-prick using a conventional blood lancet is used from a drop of fresh blood. The first end of the tube 86 and the enclosed plug/pellet 40 are brought into contact with the drop of blood, which soaks into the collagen, etc, of the plug/pellet 40.

Manual compression of the tube 86 will then push the plug/pellet 40 out of the end of the tube 86. As described in the case of the fifth device 71, it may be possible with care to deliver the plug/pellet 40 directly to where it is to be used, or it may be ejected into a container and applied conventionally.

The seventh blood collection and application device 91, shown in FIG. 9, is even simpler. This comprises an elongate rigid solid rod 92, with a plug or pellet 40 of collagen, amniotic membrane or the like attached at one end, substantially collinearly with the rod 92. This may be attached by a temporary or low-tack adhesive 90, or by a weak mechanical attachment, such as being held as a friction fit in a shallow socket at the respective end of the rod 92.

In use, a drop of fresh blood is produced by a conventional finger-prick using a conventional blood lancet. The rod 92 is held to bring the plug/pellet 40 into contact with the blood, which soaks up into the collagen, etc of the plug/pellet 40.

The plug/pellet 40 can then be disengaged from the rod 92 by simple tapping or by oblique pressure on the attachment 90 by contacting the plug/pellet 40 with tissues to be treated or with a collecting container for subsequent conventional delivery to the desired tissues.

The eighth blood collection and application device 101, shown in FIG. 10, is significantly different from the first to seventh devices 31, 41, 51, 61, 71, 81, 91 described above. In the embodiment shown, the eighth device 101 has an elongate, flat, spatulate form, its main body 102 being generally flat and parallel-sided, but with a tapering nose 103.

The eighth device 101 also works in the form of an elongate narrow cylindrical rod.

The nose 103 and adjacent portions of the body 102 have a surface that is treated or formed to pick up blood, for example by surface tension, capillary or electrostatic forces. Optionally, these surfaces may be treated with reagents such as anti-bacterials, anti-coagulants or coagulants as desired, stabilising agents, and so forth.

In use, the nose 103 is brought into contact with a drop of fresh blood, formed by conventional finger-prick methods with a conventional lancet. The blood will wick up and across the surface of the eighth device, mixing with reagents dried on the surface as appropriate. To apply the blood to tissues to be treated, the nose 103 is touched to be drawn across the tissues, depositing the blood as desired.

Further examples of products and methods embodying the present invention will now be described.

In a fourth example, a sample of whole blood was taken by finger-prick from a middle-aged patient suffering from dry eye syndrome. This blood was collected and mixed with sodium hyaluronate, as described for the first example above. The mixture was applied to the patient's eyes and compared with the results of treatment with sodium hyaluronate alone and the results of treatment with whole blood directly applied to the eye. The symptoms and signs of the dry eye syndrome improved with administration of the mixture; the improvement was greater and more long-lasting than for blood alone or sodium hyaluronate alone.

In a fifth example, a sample of whole blood was taken by finger-prick from the middle-aged patient of the fourth example. This blood was collected and mixed with hypromellose as an alternative to sodium hyaluronate. The mixture was applied to the patient's eye and compared with the results of treatment with hypromellose alone and with the results of treatment with whole blood applied directly to the eye. The symptoms and signs of the dry eye syndrome improved with administration, and the effect was greater and more long-lasting than for blood or hypromellose alone.

In a sixth example, a sample of whole blood, taken by finger-prick, was soaked into a solid cylindrical plug of collagen, around 2 mm in diameter and 5 mm in length. The plug was placed in the lower fornix of the eye of the patient of the fourth example (the fornix in this case is the pouch formed when the lower eyelid is pulled downwardly with respect to the eye, the eye forming a convex side of the pouch and the eyelid forming a concave side). When the eyelid was released, the plug was securely held between the eye and the lid, without noticeable irritation.

As the blood presumably diffused slowly out of the collagen plug, the symptoms of dry eye improved to a much greater extent than with direct administration of whole blood alone, and the improvement lasted much longer than for an equivalent amount of blood directly applied to the eye, even if applied to the fornix.

In a seventh example, finger-prick samples of blood were mixed with either the steroid dexamethasone or the NSAID diclofenac. Dexamethasone is occasionally used against dry eye irritation and inflammation, although diclofenac, while being an effective NSAID in most respects, appears not to have successfully been adopted in dry eye. Both dexamethasone with blood and diclofenac with blood, on administration to an eye of a middle-aged patient with dry-eye syndrome, led to improvements in symptoms, superior to those from blood alone. Additionally, the effects were greater than would have been expected from the anti-inflammatories alone, to the point where diclofenac was significantly effective against dry eye and levels of dexamethasone could be reduced to a quarter of the levels conventionally employed to treat dry eye and associated conditions.

In an eighth example, several drops of blood collected by finger-prick were mixed with collagen in isotonic saline. This product was gargled for a few minutes by a patient with dry mouth syndrome, and the patient's symptoms improved significantly. A collagen-based mouthwash on its own was of no benefit and added slightly to the irritation experienced as a result of dry mouth.

In a ninth example, finger-prick drops of blood were soaked into solid samples of collagen, and collagen sponge. These solid samples were placed inside the mouth of a patient with dry mouth, and the symptoms of dry mouth improved. Similar results were achieved with larger bodies of blood-soaked collagen passed around the mouth of the patient, similarly to a cough sweet or the like. Collagen sponge is a macroporous product, but still absorbs liquids such as blood into its solid structure; it is macroscopically softer and hence possibly more comfortable than solid collagen when in place. There are also a range of alternative collagens, including a wide range of physical forms, including fragmented, in aqueous suspension and in aqueous solution. Collagens from different animals are generally suitable and have a range of properties. Aquatic-derived collagen, collagen from cnidarians, and from jellyfish in particular, is found to be particularly low-irritation. Amniotic membrane is an alternative to collagen that should produce at least as good results, if not better. It is tough, resilient and capable of absorbing blood on the same basis as collagen, and there are likely to be significant residual amounts of growth factors within the amniotic membrane. Heterologous amniotic membrane and even amniotic membrane from non-human animals have been found to be compatible with human treatment.

In a tenth example, collagen gel was mixed with blood producing a relatively viscous liquid product that could be used as a mouthwash and gargled, while leaving a liquid or lightly-gelled coating on the interior of a patient's mouth. This composition was used as a mouthwash to coat an interior of the mouth of a patient with dry mouth. The symptoms of dry mouth improved greatly. The viscous product, lingering within the mouth like some linctuses and syrups, maintained the benefits for longer than a simple wash with blood and saline. As noted above for the eighth example, the collagen alone would have been of no benefit or would have added to the irritation.

In the eleventh, twelfth and thirteenth examples, blood was mixed with a composition comprising a lubricant, hyaluronic acid, an antimicrobial agent, collagen gel and a clotting agent. This produced a viscous composition suitable for coating a range of further epithelial surfaces. In the eleventh example, the composition was applied to dry and broken skin and nails; in the twelfth example, the composition was applied to a bald patch on the scalp and to a hairless patch on the face; in the thirteenth example, it was applied to wrinkles in the skin of the face, eyelids and forehead. It is expected that at least cosmetic improvements will result, although insufficient time has yet passed to be certain. Parts of blood, such as serum or growth factors are expected to work equally well.

In a fourteenth example, blood or parts of blood were mixed with a lubricant, hyaluronic acid, an antimicrobial agent, collagen or collagen gel and a clotting agent. This formed a gelled, hydrated composition that was applied to a skin ulcer on a patient's leg as a dressing or bandage, to speed healing of the ulcerated tissues.

In a fifteenth example, blood or parts of blood would again be mixed with a lubricant, hyaluronic acid, an antimicrobial agent, collagen or collagen gel and a clotting agent. This composition would be suitable for treating other epithelial surfaces, particularly ciliary epithelium, such as forms the surface of the lungs, gut, the organ of Corti and the remainder of the inner ear. For the lungs, for example, a spray or mist would be the best delivery vector, from an inhaler similar to an asthma inhaler.

In a sixteenth example, blood or parts of blood would again be mixed with a lubricant, hyaluronic acid, an antimicrobial agent, collagen or collagen gel and a clotting agent. This composition would be applicable to any skin condition, especially if formulated to behave as an ointment or gel.

In a seventeenth example, a similar composition containing parts of blood, ideally concentrated platelets or isolated growth factors, would be made up with a low viscosity so as to be locally injectable, possible in parts of the body where blood supply is poor.

In an eighteenth example, a mouth wash was formulated using blood serum. When this is gargled by a patient having dry-mouth, his symptoms would improve.

In nineteenth and twentieth examples, blood plasma was used in place of whole blood in an eye drops and a mouth wash formulation as described above, respectively. These, too, improved the symptoms of dry eye and dry mouth respectively.

In a twenty-first example, dehydrated blood plasma or serum (both spray-dried and lyophilised/freeze-dried) were reconstituted and used in place of fresh plasma or serum respectively in formulations and tests of respective examples above. This gave similarly successful results.

Other approaches to delivering blood or blood parts/products to epithelial cells include sprays, ointments, gels, longer-acting formulations such as slow-release systems, and so on. The collagen plugs described in the sixth example above would, if sized suitably, be suitable as implants, and particularly as punctal plugs, located in the tear ducts, or salivary duct plugs, which have both been found to be useful treatment vectors for dry eye and potentially should be for dry mouth.

One interesting point about using collagen as a reservoir for blood and the like is that there is a natural reaction between collagen and blood platelets, as part of wound healing. It is therefore believed that the blood cells and platelets held in the collagen plugs, rods, pellets, etc, might well be reacting slowly with the collagen, possibly releasing growth factors by breaching cell membranes.

With collagen and amniotic membrane both being natural products, it is believed that implantation in a wide range of locations around the body would be feasible, such and intra oral, dental, in and around the ear, adjacent the gut and so forth.

As an alternative to collagen and amniotic membrane, it is believed that silicones would be effective, particularly to hold and release growth factors; plain punctal plugs of silicone are known, and silicones can be used to form slow-release implants in general.

While the above examples have concentrated on the use of human blood and hence growth factors in treating humans, the same approaches should work for non-human animals.

Although autologous blood and blood parts may be preferable in some instances, other applications, such as around the mouth, will probably permit the use of heterologous materials and even material from non-human animals. Serums should be particularly flexible in applicability.

It should be noted that in the context of this application, the term blood part or blood product should be understood to include one of more growth factors isolable from blood. The following growth factors and the like are believed to be highly beneficial (while still very expensive in isolated form):

### Adrenomedullin (AM)

### Angiopoietin (Mg)

Autocrine motility factor
Bone morphogenetic proteins (BMPs)
Brain-derived neurotrophic factor (BDNF)
Epidermal growth factor (EGF)

### Erythropoietin (EPO)

Fibroblast growth factor (FGF)

### Foetal Bovine Somatotrophin (FBS)

Glial cell line-derived neurotrophic factor (GDNF)
Granulocyte colony-stimulating factor (G-CSF)
Granulocyte macrophage colony-stimulating factor (GM-CSF)
Growth differentiation factor-9 (GDF9)
Hepatocyte growth factor (HGF)
Hepatoma-derived growth factor (HDGF)
Insulin-like growth factor (IGF)
Keratinocyte growth factor (KGF)
Migration-stimulating factor (MSF)

### Myostatin (GDF-8)

Nerve growth factor (NGF) and other neurotrophins
Platelet-derived growth factor (PDGF) (“Healing Factor)

### Thrombopoietin (TPO)

T-cell growth factor (TCGF)
Transforming growth factor alpha(TGF-α)
Transforming growth factor beta(TGF-β)
Tumor necrosis factor-alpha(TNF-α)
Vascular endothelial growth factor (VEGF)

### Wnt Signaling Pathway

Placental growth factor (PGF)
IL-1—Cofactor for IL-3 and IL-6. Activates T cells.
IL-2—T-cell growth factor. Stimulates IL-1 synthesis. Activates B-cells and NK cells.
IL-3—Stimulates production of all non-lymphoid cells.
IL-4—Growth factor for activated B cells, resting T cells, and mast cells.
IL-5—Induces differentiation of activated B cells and eosinophils.
IL-6—Stimulates Ig synthesis. Growth factor for plasma cells.
IL-7—Growth factor for pre-B cells.
Renalase—RNLS—Anti-apoptotic survival factor

1. A treatment element for treating disorders of epithelial tissues of the human or non-human animal body, comprising solid reservoir means of collagen or amniotic membrane containing a dose of blood or a blood product.

2. A treatment element as claimed in claim 1, for treatment of a particular human or non-human animal patient, wherein said blood comprises autologous whole blood from said patient.

3. A treatment element as claimed in claim 1, for treatment of a non-specific patient, wherein said blood product comprises blood serum.

4. A treatment element as claimed in claim 1, wherein the blood product comprises one or more growth factors isolable from blood.

5. A treatment element as claimed in any one of the preceding claims, for treatment of tissues of the eye, wherein the reservoir means is adapted to be located in the lower fornix of the eye of a patient.

6. A treatment element as claimed in claim 5, wherein the reservoir means comprises generally cylindrical rod means, less than 15 millimetres in length and less than 4 millimetres in diameter.

7. A treatment element as claimed in claim 6, wherein the generally cylindrical rod means is no more than 5 millimetres in length and no more than 2 millimetres in diameter.

8. A treatment element as claimed in any one of claims 1 to 4, for treatment of tissues of the eye, wherein the reservoir means comprises a punctal plug, adapted for insertion into a tear duct.

9. A treatment element as claimed in claim 8, wherein the reservoir means comprises generally cylindrical rod means, less than 3 millimetres in length and less than 1 millimetre in diameter.

10. A treatment element as claimed in any one of claims 1 to 4, for treatment of tissues of the mouth, wherein the reservoir means has the form of generally spheroidal or ellipsoidal lozenge means.

11. A treatment element as claimed in claim 10, wherein said lozenge means is between 5 and 15 millimetres in average diameter.

12. A treatment element as claimed in either claim 10 or claim 11, for treatment of dry mouth.

13. A treatment element as claimed in any one of claims 1 to 4, for treatment of tissues of the mouth, wherein the reservoir means has the form of a pad or patch contactable with said tissues.

14. A treatment element as claimed in claim 13, wherein the reservoir means is generally laminar with a maximum thickness of less than 5 millimetres, optionally less than 3 millimetres.

15. A treatment element as claimed in either claim 13 or 14, for treatment of mouth ulcers, the reservoir means being configured to cover an entire area of ulceration.

16. A treatment element as claimed in either claim 13 or claim 14, wherein the reservoir means is configured to cover a single ulcerated lesion.

17. A treatment element as claimed in any one of claims 1 to 4, for treatment of skin tissues, wherein the reservoir means has the form of a dressing or bandage.

18. A treatment element as claimed in claim 17, wherein the reservoir means is generally laminar with a maximum thickness of less than 3 millimetres, optionally less than 2 millimetres.

19. A treatment element according to any one of the preceding claims, wherein said collagen comprises collagen of non-human origin.

20. A treatment element according to any one of claims 1 to 18, wherein said collagen comprises collagen from a marine organism, optionally derived from jellyfish or other cnidarians.

21. A treatment element according to any one of claims 1 to 18, wherein aid collagen comprises fragmented collagen or hydrolysed collagen.

22. A treatment element according to any one of claims 1 to 18, wherein said collagen comprises collagen sponge or collagen gel.

23. A treatment element according to any one of claims 1 to 18, wherein said amniotic membrane comprises amniotic membrane of non-human origin.

24. A method of treatment for disorders of epithelial tissues of the human or non-human animal body, comprising the steps of providing solid reservoir means of collagen or amniotic membrane; providing a dose of blood or blood product; absorbing said dose into the reservoir means; positioning the reservoir means adjacent the tissues to be treated; and allowing the blood or blood product to diffuse or disperse out of the reservoir means to contact said tissues.

25. A method of treatment as claimed in claim 24, comprising treatment of a particular human or non-human animal patient, said dose of blood comprising autologous whole blood.

26. A method of treatment as claimed in claim 24, comprising treatment of a non-specific patient, wherein said dose of blood product comprises blood plasma or serum, preferably serum.

27. A composition for the treatment of epithelial disorders for a particular human or non-human animal patient, comprising autologous whole blood extracted from said patient, mixed with an aqueous carrier medium.

28. A composition as claimed in claim 27, adapted for the treatment of ophthalmic disorders.

29. A composition as claimed in claim 28, wherein the aqueous carrier medium comprises an eyedrops formulation.

30. A composition as claimed in claim 28, wherein the aqueous carrier medium may comprises an artificial tears composition.

31. A method for the treatment of epithelial disorders of humans or non-human animals, comprising the steps of extracting a quantity of whole blood from a patient requiring treatment for said disorder, mixing said whole blood with an aqueous carrier solution to form a treatment composition and administering said treatment composition to or adjacent disordered epithelial tissues of the patient.

32. A method of treatment as claimed in claim 31, wherein the method is for treatment of disorders of the eye.

33. A method of treatment as claimed in claim 32, wherein said treatment composition comprises an artificial tears or eyedrops composition, and the application step comprises instilling the composition on to a surface of the eye.

34. A composition for the treatment of epithelial disorders for a human or non-human animal patient, comprising growth factors or proteins, mixed with an aqueous carrier medium.

35. A method for the treatment of epithelial disorders of humans or non-human animals, comprising the steps of providing one or more growth factors or proteins, mixing said growth factors or proteins with an aqueous carrier solution to form a treatment composition, and administering said treatment composition to or adjacent disordered epithelial tissues of the patient.

36. A composition for the treatment of disorders of epithelial tissues of the mouth for a human or non-human animal patient, comprising blood or parts of the blood or blood products incorporated into a preparation adapted for application to the epithelial tissues of the mouth.

37. A method of treatment for disorders of the epithelial tissues of the mouth for a human or non-human animal patient, comprising the steps of providing a composition as claimed in claim 36 and applying it to an interior of the mouth so as to contact the tissues to be treated.

38. A composition for the treatment of disorders of epithelial tissues for a human or non-human animal patient, comprising dehydrated blood or parts of the blood from which water has been partially or totally removed.

39. A blood collection and application device, the device comprising:
flexible wall means defining internally chamber means containing an aqueous carrier solution miscible with blood or a blood product to form a treatment composition, and means to introduce said bodily fluid into said chamber means for mixing.

40. A blood collection and application device, the device comprising:
elongate outer casing means including a resiliently compressible portion, the casing means defining internally a cavity; and
flexible chamber means located within said cavity, and comprising tube means extending from said chamber means outwardly through said outer casing means, said tube means communicating said chamber means with a surrounding environment; in which
said flexible chamber means is movable along said cavity between a first position and a second position, wherein in said second position a majority of the tube means is disposed within the outer casing means and wherein in said first position, said flexible chamber is positioned proximal said resiliently compressible portion of said outer casing.

41. A blood collection and application device, the device comprising:
hollow elongate rigid tube means having a first open end and a second end, and having pellet means of collagen or amniotic membrane located within said tube means adjacent said first open end; and
plunger means located within said tube means comprising a head portion and a shaft portion, said head portion being contactable with said pellet means and said shaft portion extending outwardly from said second end of said tube; in which
said plunger means is slidable within said tube means, from a first position towards a second position, and wherein in said second position, said head portion causes said pellet means to be displaced from within said tube.

42. A blood collection and application device, the device comprising:
hollow elongate compressible tube means having a first open end and a second closed end, and having pellet means of collagen or amniotic membrane located within said tube means adjacent said first open end; in which
compression of said compressible tube means urges said pellet to eject from within said tube means.

43. A blood collection and application device, the device comprising:
elongate rigid rod means having a first end and a second end, the rod means having pellet means of collagen or amniotic membrane releasably attached proximal said first end; in which said pellet means is disengageable from said rod by an external force incident said rod means or said pellet means.

44. A blood collection and application device, the device comprising:
an elongate main body defining an adsorption surface, in which said adsorption surface is so treated or configured as to attract blood to flow on to said adsorption surface.
